(19) United States
(12) Patent Application Publication
(10) Pub. No.: US 2017/0281411 A1
(43) Pub. Date: Oct. 5, 2017

(54) SINGLE-USE LASER PROBE WITH REUSABLE OPTIC FIBER FIXTURES

(71) Applicant: Katalyst Surgical, LLC, Chesterfield, MO (US)

(72) Inventors: Gregg D. Scheller, Wildwood, MO (US); Matthew N. Yates, High Ridge, MO (US)

(73) Assignee: Katalyst Surgical, LLC, Chesterfield, MO (US)

(21) Appl. No.: 15/460,700

(22) Filed: Mar. 16, 2017

Related U.S. Application Data
(60) Provisional application No. 62/317,195, filed on Apr. 1, 2016.

(51) Int. Cl. A61F 9/008; A61B 90/30; A61B 18/22

(52) U.S. Cl. CPC A61F 9/00821; A61B 18/22; A61B 90/30; A61B 2017/00477

(57) ABSTRACT

A single-use laser probe with reusable optic fiber fixtures may include a transitory connector having a transitory connector distal end and a transitory connector proximal end; a tube having a tube distal end and a tube proximal end; and an optic fiber having an optic fiber distal end and an optic fiber proximal end. The optic fiber may be disposed in the transitory connector wherein the optic fiber proximal end extends a distance from the transitory connector distal end. The optic fiber may be disposed in the tube wherein the optic fiber distal end is coplanar with the tube distal end. The transitory connector may be configured to interface with a reusable optic fiber fixture.

### CROSS-REFERENCE TO RELATED APPLICATIONS

This Application claims the benefit of U.S. Provisional Application No. 62/317,195, filed Apr. 1, 2016.

### FIELD OF THE INVENTION

The present disclosure relates to a surgical instrument, and, more particularly, to a single-use laser probe with reusable optic fiber fixtures.

### BACKGROUND OF THE INVENTION

A wide variety of ophthalmic procedures require a laser energy source. For example, ophthalmic surgeons may use laser photocoagulation to treat proliferative retinopathy. Proliferative retinopathy is a condition characterized by the development of abnormal blood vessels in the retina that grow into the vitreous humor. Ophthalmic surgeons may treat this condition by energizing a laser to cauterize portions of the retina to prevent the abnormal blood vessels from growing and hemorrhaging. Typically, treatments are performed using a disposable, single-use laser probe connected to a laser surgical machine by an optical fiber. Unfortunately, use of disposable, single-use laser probes increases treatment costs because a new laser probe is required for each surgical treatment. Accordingly, there is a need for a laser probe that may be safely used to perform more than one surgical procedure.

### BRIEF SUMMARY OF THE INVENTION

The present disclosure provides a single-use laser probe with reusable optic fiber fixtures. In one or more embodiments, a single-use laser probe with reusable optic fiber fixtures may comprise a transitory connector having a transitory connector distal end and a transitory connector proximal end; a tube having a tube distal end and a tube proximal end; and an optic fiber having an optic fiber distal end and an optic fiber proximal end. Illustratively, the optic fiber may be disposed in the transitory connector wherein the optic fiber proximal end extends a distance from the transitory connector distal end. In one or more embodiments, the optic fiber may be disposed in the tube wherein the optic fiber distal end is coplanar with the tube distal end. Illustratively, the transitory connector may be configured to interface with a reusable optic fiber fixture.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A and 1B are schematic diagrams illustrating a transitory connector;

FIG. 2 is a schematic diagram illustrating an exploded view of a single-use laser probe assembly;

FIGS. 3A and 3B are schematic diagrams illustrating an assembled single-use laser probe;

FIG. 4 is a schematic diagram illustrating an exploded view of an optic fiber fixture assembly;

FIGS. 5A and 5B are schematic diagrams illustrating an assembled optic fiber fixture;

FIGS. 6A and 6B are schematic diagrams illustrating a single-use laser probe with a reusable optic fiber fixture;

FIGS. 7A and 7B are schematic diagrams illustrating a one-piece handle;

FIGS. 8A and 8B are schematic diagrams illustrating a single-use one-piece laser probe with a reusable optic fiber fixture;

FIG. 9 is a schematic diagram illustrating an exploded view of a single-use illuminated laser probe assembly;

FIGS. 10A and 10B are schematic diagrams illustrating an assembled single-use illuminated laser probe;

FIGS. 11A and 11B are schematic diagrams illustrating an illumination optic fiber fixture;

FIGS. 12A and 12B are schematic diagrams illustrating a single-use illuminated laser probe with a reusable optic fiber fixture and a reusable illumination optic fiber fixture.

### DETAILED DESCRIPTION OF AN ILLUSTRATIVE EMBODIMENT

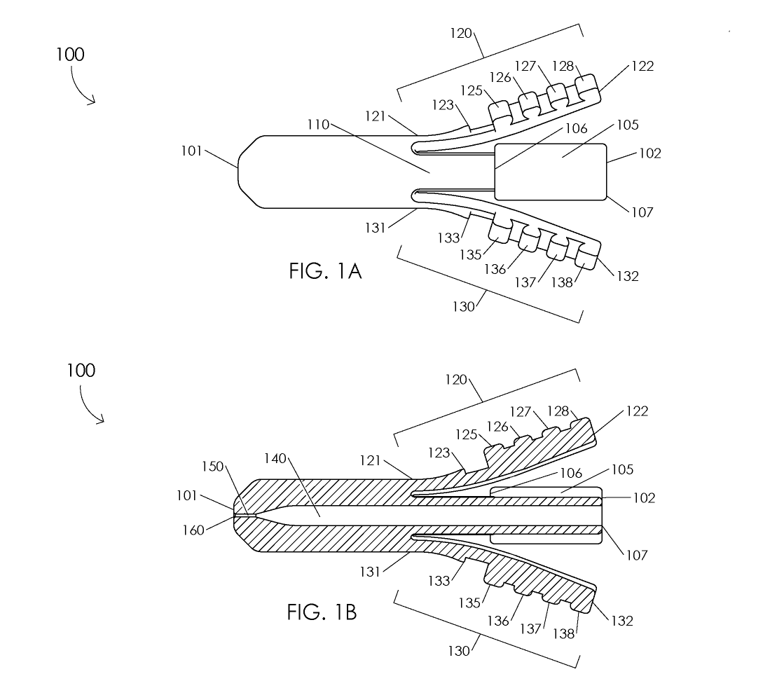

FIGS. 1A and 1B are schematic diagrams illustrating a transitory connector 100. FIG. 1A illustrates a side view of a transitory connector 100. Illustratively, transitory connector 100 may comprise a transitory connector distal end 101, a transitory connector proximal end 102, a proximal base 105, a distal base 110, a superior arm 120, and an inferior arm 130. In one or more embodiments, proximal base 105 may comprise a proximal base distal end 106 and a proximal base proximal end 107. Illustratively, distal base 110 may be disposed between transitory connector distal end 101 and proximal base 105, e.g., distal base 110 may be disposed between transitory connector distal end 101 and proximal base distal end 106. In one or more embodiments, proximal base proximal end 107 may be transitory connector proximal end 102. Illustratively, superior arm 120 may comprise a superior arm distal end 121 and a superior arm proximal end 122. In one or more embodiments, superior arm 120 may comprise a superior arm barb 123, e.g., superior arm 120 may comprise a superior arm barb 123 disposed between superior arm distal end 121 and superior arm proximal end 122. Illustratively, superior arm 120 may comprise a first lateral projection 125, a second lateral projection 126, a third lateral projection 127, and a fourth lateral projection 128. In one or more embodiments, first lateral projection 125 may be disposed between superior arm barb 123 and second lateral projection 126. Illustratively, second lateral projection 126 may be disposed between first lateral projection 125 and third lateral projection 127. In one or more embodiments, third lateral projection 127 may be disposed between second lateral projection 126 and fourth lateral projection 128. Illustratively, fourth lateral projection 128 may be disposed between third lateral projection 127 and superior arm proximal end 122. In one or more embodiments, superior arm 120 may be disposed between transitory connector distal end 101 and transitory connector proximal end 102. Illustratively, inferior arm 130 may comprise an inferior arm distal end 131 and an inferior arm proximal end 132. In one or more embodiments, inferior arm 130 may comprise an inferior arm barb 133, e.g., inferior arm 130 may comprise an inferior arm barb 133 disposed between inferior arm distal end 131 and inferior arm proximal end 132. Illustratively, inferior arm 130 may comprise a first lateral projection 135, a second lateral projection 136, a third lateral projection 137, and a fourth lateral projection 138. In one or more embodiments, first lateral projection 135 may be disposed between inferior arm barb 133 and second lateral projection 136. Illustratively, second lateral projection 136 may be disposed between first lateral projection 135 and third lateral projection 137. In one or more embodiments, third lateral projection 137 may be disposed between second lateral projection 136 and fourth lateral projection 138. Illustratively, fourth lateral projection 138 may be disposed between third lateral projection 137 and inferior arm proximal end 132. In one or more embodiments, inferior arm 130 may be disposed between transitory connector distal end 101 and transitory connector proximal end 102.

FIG. 1B illustrates a cross-sectional view in a sagittal plane of a transitory connector 100. Illustratively, transitory connector 100 may comprise a tapered inner lumen 140, an optic fiber housing 150, and a fixation mechanism housing 160. In one or more embodiments, transitory connector 100 may be manufactured from a material configured to deform if transitory connector 100 is sterilized in a medical autoclave, e.g., transitory connector 100 may be manufactured from a material configured to permanently deform if transitory connector 100 is sterilized in a medical autoclave. Illustratively, transitory connector 100 may be manufactured from a material having a melting point below a temperature parameter for a steam sterilization cycle, e.g., transitory connector 100 may be manufactured from a material having a melting point below a temperature parameter for a gravity-displacement steam sterilization cycle, a dynamic-air-removal steam sterilization cycle, etc. In one or more embodiments, transitory connector 100 may be manufactured from a material having a melting point below 140.0 degrees Fahrenheit. Illustratively, transitory connector 100 may be manufactured from a material having a melting point in a range of 158.0 to 212.0 degrees Fahrenheit, e.g., transitory connector 100 may be manufactured from a material having a melting point of 160.0 degrees Fahrenheit. In one or more embodiments, transitory connector 100 may be manufactured from a material having a melting point of less than 158.0 degrees Fahrenheit or greater than 212.0 degrees Fahrenheit. In one or more embodiments, transitory connector 100 may be manufactured from a material having a melting point below 250.0 degrees Fahrenheit. Illustratively, transitory connector 100 may be manufactured from a material having a melting point below 270.0 degrees Fahrenheit. In one or more embodiments, transitory connector 100 may be manufactured from a material having a melting point below 275.0 degrees Fahrenheit.

Illustratively, transitory connector 100 may be manufactured from a material configured to temporarily deform if transitory connector 100 is sterilized in a medical auto-clave, e.g., transitory connector 100 may be manufactured from a material configured to absorb water in a medical autoclave. In one or more embodiments, an absorption of water may be configured to deform transitory connector 100, e.g., an absorption of water may be configured to cause transitory connector 100 to expand. Illustratively, transitory connector 100 may be manufactured from a porous material configured to facilitate a deformation of transitory connector 100 if transitory connector 100 is sterilized in a medical autoclave. In one or more embodiments, transitory connector 100 may be manufactured with one or more cavities configured to facilitate a deformation of transitory connector 100 if transitory connector 100 is sterilized in a medical autoclave. Illustratively, transitory connector 100 may be manufactured from any suitable material, e.g., polymers, metals, metal alloys, etc., or from any combination of suitable materials. In one or more embodiments, transitory connector 100 may be manufactured by a 3D printing process. For example, transitory connector 100 may be manufactured by selective laser sintering, selective heat sintering, selective laser melting, electron-beam melting, direct metal laser sintering, electron beam freeform fabrication, etc. Illustratively, transitory connector 100 may be manufactured by injection molding.

In one or more embodiments, transitory connector 100 may be manufactured from poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(capryl aldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene pi-perazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene di siloxanylenedi-propionamide), poly[(ethylenedithio)-diacetic anhydride], poly[(ethylenedithio)-dipropionic anhydride], poly(ethylene dithionisophthalate), poly(ethelene dithiotetra-methylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc. Illustratively, transitory connector 100 may be manufactured from any substituted polymers of poly(acrylamide), poly(acrylic acid), poly(adipic is anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(capryl aldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene di siloxanylenedi-propionamide), poly[(ethylenedithio)-diacetic anhydride], poly[(ethylenedithio)-dipropionic anhydride], poly(ethylene dithionisophthalate), poly(ethelene dithiotetra-methylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc.

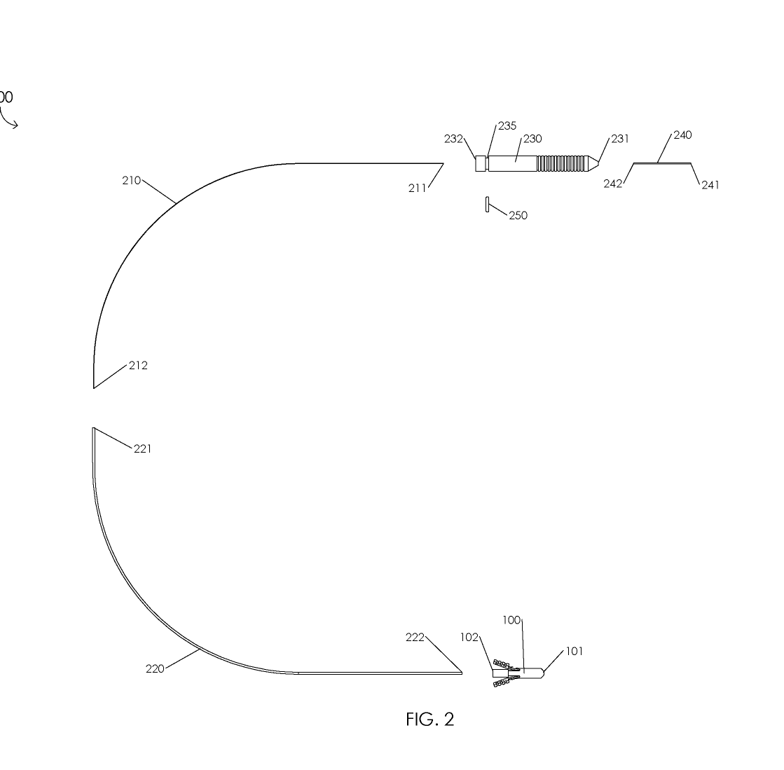

FIG. 2 is a schematic diagram illustrating an exploded view of a single-use laser probe assembly 200. Illustratively, a single-use laser probe assembly 200 may comprise a transitory connector 100, an optic fiber 210, a jacketing 220, a handle 230, a hypodermic tube 240, and an identification ring 250. In one or more embodiments, optic fiber 210 may comprise an optic fiber distal end 211 and an optic fiber proximal end 212. Illustratively, optic fiber 210 may be configured to transmit laser light. In one or more embodiments, jacketing 220 may comprise a jacketing distal end 221 and a jacketing proximal end 222. Illustratively, jacketing 220 may be manufactured from any suitable material, e.g., polymers, metals, metal alloys, etc., or from any combination of suitable materials. In one or more embodiments, handle 230 may comprise a handle distal end 231, a handle proximal end 232, and an identification ring channel 235. Illustratively, hypodermic tube 240 may comprise a hypodermic tube distal end 241 and a hypodermic tube proximal end 242. In one or more embodiments, hypodermic tube 240 may be manufactured from any suitable material, e.g., polymers, metals, metal alloys, etc., or from any combination of suitable materials.

In one or more embodiments, handle 230 may be manufactured from a material is configured to deform if handle 230 is sterilized in a medical autoclave, e.g., handle 230 may be manufactured from a material configured to permanently deform if handle 230 is sterilized in a medical autoclave. Illustratively, handle 230 may be manufactured from a material having a melting point below a temperature parameter for a steam sterilization cycle, e.g., handle 230 may be manufactured from a material having a melting point below a temperature parameter for a gravity-displacement steam sterilization cycle, a dynamic-air-removal steam sterilization cycle, etc. In one or more embodiments, handle 230 may be manufactured from a material having a melting point below 140.0 degrees Fahrenheit. Illustratively, handle 230 may be manufactured from a material having a melting point in a range of 158.0 to 212.0 degrees Fahrenheit, e.g., handle 230 may be manufactured from a material having a melting point of 160.0 degrees Fahrenheit. In one or more embodiments, handle 230 may be manufactured from a material having a melting point of less than 158.0 degrees Fahrenheit or greater than 212.0 degrees Fahrenheit. In one or more embodiments, handle 230 may be manufactured from a material having a melting point below 250.0 degrees Fahrenheit. Illustratively, handle 230 may be manufactured from a material having a melting point below 270.0 degrees Fahrenheit. In one or more embodiments, handle 230 may be manufactured from a material having a melting point below 275.0 degrees Fahrenheit.

Illustratively, handle 230 may be manufactured from a material configured to temporarily deform if handle 230 is sterilized in a medical autoclave, e.g., handle 230 may be manufactured from a material configured to absorb water in a medical autoclave. In one or more embodiments, an absorption of water may be configured to deform handle 230, e.g., an absorption of water may be configured to cause handle 230 to expand. Illustratively, handle 230 may be manufactured from a porous material configured to facilitate a deformation of handle 230 if handle 230 is sterilized in a medical autoclave. In one or more embodiments, handle 230 may be manufactured with one or more cavities configured to facilitate a deformation of handle 230 if handle 230 is sterilized in a medical autoclave. Illustratively, handle 230 may be manufactured from any suitable material, e.g., polymers, metals, metal alloys, etc., or from any combination of suitable materials. In one or more embodiments, handle 230 may be manufactured by a 3D printing process. For example, handle 230 may be manufactured by selective laser sintering, selective heat sintering, selective laser melting, electron-beam melting, direct metal laser sintering, electron beam freeform fabrication, etc. Illustratively, handle 230 may be manufactured by injection molding.

In one or more embodiments, handle 230 may be manufactured from poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene pi-perazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene di siloxanylenedi-propionamide), poly[(ethylenedithio)-diacetic anhydride], poly[(ethylenedithio)-dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydi-acetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc. Illustratively, handle 230 may be manufactured from any substituted polymers of poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)-dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc.

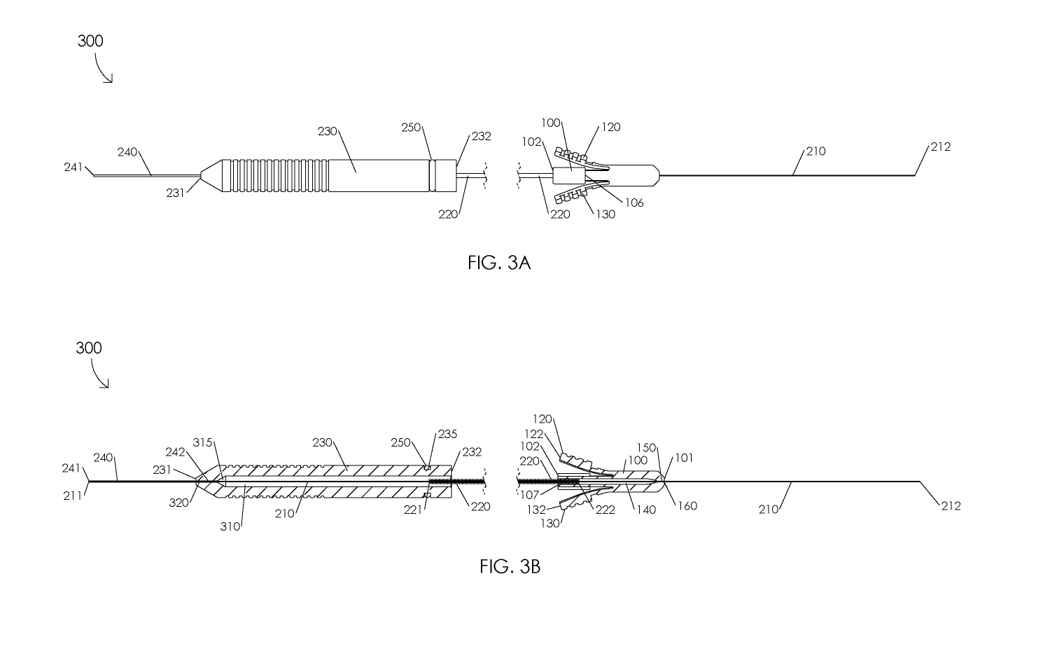

FIGS. 3A and 3B are schematic diagrams illustrating an assembled single-use laser probe 300. FIG. 3A illustrates a side view of an assembled single-use laser probe 300. Illustratively, optic fiber 210 may be disposed in transitory connector 100 wherein optic fiber proximal end 212 extends a distance from transitory connector distal end 101. In one or more embodiments, identification ring 250 may be disposed over a portion of handle 230, e.g., identification ring 250 may be disposed in identification ring channel 235. Illustratively, identification ring 250 may be configured to visually indicate one or more properties of an assembled single-use laser probe 300, e.g., identification ring 250 may be configured to indicate a compatible cannula gauge size for insertion of hypodermic tube 240. In one or more embodiments, identification ring 250 may be configured to visually indicate if an assembled single-use laser probe 300 has been used in a surgical procedure, e.g., identification ring 250 may be configured to change from a first color to a second color when sterilized in a medical autoclave after a use in a surgical procedure.

FIG. 3B illustrates a cross-sectional view in a sagittal plane of an assembled single-use laser probe 300. In one or more embodiments, an assembled single-use laser probe 300 may comprise an inner bore 310, an inner bore distal taper 315, and a hypodermic tube housing 320. Illustratively, a portion of hypodermic tube 240 may be disposed in a portion of handle 230, e.g., hypodermic tube proximal end 242 may be disposed in a portion of handle 230. In one or more embodiments, hypodermic tube proximal end 242 may be disposed in hypodermic tube housing 320 wherein hypodermic tube distal end 241 extends a distance from handle distal end 231. Illustratively, a portion of hypodermic tube 240 may be fixed within a portion of handle 230, e.g., a portion of hypodermic tube 240 may be fixed within a portion of handle 230 by a friction fit, an adhesive, a crimp, a weld, etc.

Illustratively, optic fiber 210 may be disposed in jacketing 220, e.g., optic fiber 210 may be disposed in jacketing 220 wherein one or more portions of optic fiber 210 extend from one or more portions of jacketing 220. In one or more embodiments, optic fiber 210 may be disposed in jacketing 220 wherein optic fiber distal end 211 extends a distance from jacketing distal end 221. Illustratively, optic fiber 210 may be disposed in jacketing 220 wherein optic fiber proximal end 212 extends a distance from jacketing proximal end 222. In one or more embodiments, a portion of jacketing 220 may be disposed in a portion of transitory connector 100, e.g., jacketing proximal end 222 may be disposed in a portion of transitory connector 100. Illustratively, jacketing proximal end 222 may be disposed in tapered inner lumen 140. In one or more embodiments, a portion of jacketing 220 may be fixed within a portion of transitory connector 100, e.g., a portion of jacketing 220 may be fixed within a portion of transitory connector 100 by a friction fit, an adhesive, a crimp, a weld, etc. Illustratively, a portion of jacketing 220 may be disposed in a portion of handle 230, e.g., jacketing distal end 221 may be disposed in a portion of handle 230. In one or more embodiments, jacketing distal end 221 may be disposed in inner bore 310. Illustratively, a portion of jacketing 220 may be fixed within a portion of handle 230, e.g., a portion of jacketing 220 may be fixed within a portion of handle 230 by a friction fit, an adhesive, a crimp, a weld, etc.

In one or more embodiments, a portion of optic fiber 210 may be disposed in transitory connector 100, e.g., optic fiber 210 may be disposed in tapered inner lumen 140, optic fiber housing 150, and fixation mechanism housing 160. Illustratively, optic fiber 210 may be fixed in a position relative to transitory connector 100, e.g., optic fiber 210 may be fixed within transitory connector 100 wherein optic fiber proximal end 212 extends a distance from transitory connector distal end 101. In one or more embodiments, a portion of optic fiber 210 may be fixed within a portion of transitory connector 100, e.g., a portion of optic fiber 210 may be fixed within a portion of transitory connector 100 by a friction fit, an adhesive, a crimp, a weld, etc. Illustratively, a fixation mechanism may be configured to fix a portion of optic fiber 210 within optic fiber housing 150, e.g., a fixation mechanism may be disposed in fixation mechanism housing 160 wherein the fixation mechanism is configured to fix a portion of optic fiber 210 within optic fiber housing 150.

In one or more embodiments, a portion of optic fiber 210 may be disposed in handle 230 and hypodermic tube 240, e.g., optic fiber 210 may be disposed in inner bore 310, inner bore distal taper 315, and hypodermic tube housing 320. Illustratively, optic fiber 210 may be disposed within handle 230 and hypodermic tube 240 wherein optic fiber distal end 241 is adjacent to hypodermic tube distal end 241. In one or more embodiments, optic fiber 240 may be disposed within handle 230 and hypodermic tube 240 wherein optic fiber distal end 241 is coplanar with hypodermic tube distal end 241. Illustratively, hypodermic tube distal end 241 may comprise a tapered portion configured to prevent degradation to a laser spot wherein optic fiber distal end 211 is recessed from hypodermic tube distal end 241 relative to a laser spot wherein optic fiber distal end 211 is coplanar with hypodermic tube distal end 241. In one or more embodiments, hypodermic tube distal end 241 may comprise a tapered portion configured to ensure that a power output wherein optic fiber distal end 211 is recessed relative to hypodermic tube distal end 241 is identical to a power output wherein optic fiber distal end 211 is coplanar with hypodermic tube distal end 241. Illustratively, hypodermic tube distal end 241 may comprise a tapered portion configured to ensure that a laser spot size wherein optic fiber distal end 211 is recessed relative to hypodermic tube distal end 241 is identical to a laser spot size wherein optic fiber distal end 211 is coplanar with hypodermic tube distal end 241. In one or more embodiments, hypodermic tube distal end 241 may comprise a tapered portion configured to ensure that a laser spot shape wherein optic fiber distal end 211 is recessed relative to hypodermic tube distal end 241 is identical to a laser spot shape wherein optic fiber distal end 211 is coplanar with hypodermic tube distal end 241. Illustratively, optic fiber 210 may be fixed within hypodermic tube 240, e.g., optic fiber 210 may be fixed within hypodermic tube 240 by a friction fit, an adhesive, a crimp, a weld, etc. In one or more embodiments, optic fiber 210 may be fixed within handle 230, e.g., optic fiber 210 may be fixed within handle 230 by a friction fit, an adhesive, a crimp, a weld, etc.

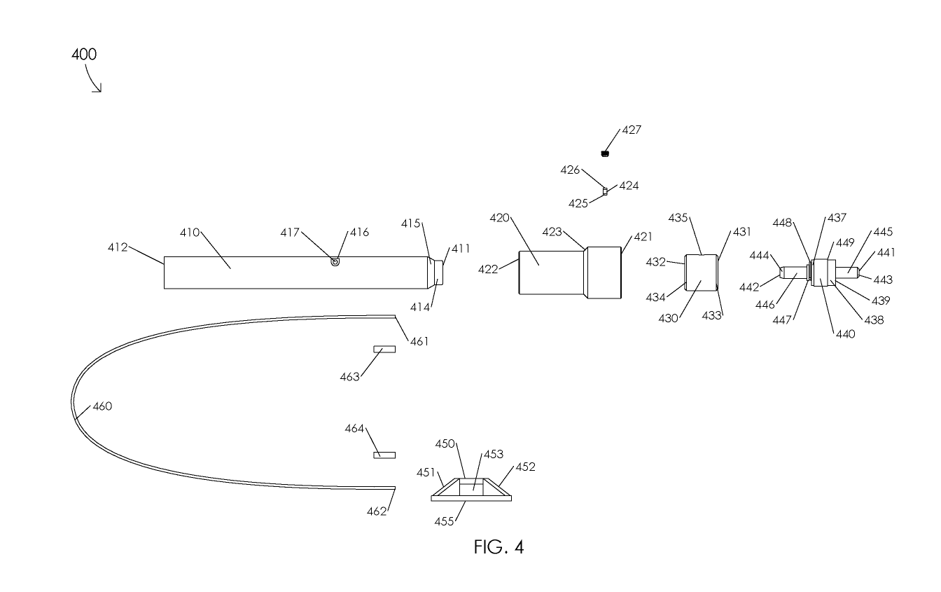

FIG. 4 is a schematic diagram illustrating an exploded view of an optic fiber fixture assembly 400. Illustratively, an optic fiber fixture assembly 400 may comprise a fixture base 410, a machine connector housing 420, an electrical element 424, a fixation mechanism 427, an extender 430, a machine connector 440, a machine coupler 450, a lanyard cable 460, a distal fastener 463, and a proximal fastener 464. In one or more embodiments, fixture base 410 may comprise a fixture base distal end 411 and a fixture base proximal end 412. Illustratively, fixture base 410 may comprise an extender interface 414, a fixture base distal taper 415, an indentation 416, and a lanyard cable guide 417. In one or more embodiments, lanyard cable guide 417 may be disposed in indentation 416. Illustratively, lanyard cable guide 417 may be disposed dorsally in fixture base 410. In one or more embodiments, machine connector housing 420 may comprise a machine connector housing distal end 421 and a machine connector housing proximal end 422. Illustratively, machine connector housing 420 may comprise a machine connector housing taper 423. In one or more embodiments, extender 430 may comprise an extender distal end 431 and an extender proximal end 432. Illustratively, extender 430 may comprise an extender distal taper 433 and an extender proximal taper 434. In one or more embodiments, extender 430 may comprise an electrical element housing 435. Illustratively, electrical element housing 435 may be configured to house electrical element 424. In one or more embodiments, electrical element 424 may comprise an electrical element inferior end 425 and an electrical element superior end 426.

In one or more embodiments, machine connector 440 may comprise a machine connector distal end 441 and a machine connector proximal end 442. Illustratively, machine connector 440 may comprise a machine connector distal taper 443, a machine connector proximal taper 444, a distal ferrule 445, and a proximal ferrule 446. In one or more embodiments, machine connector 440 may comprise a machine connector base 438. Illustratively, machine connector base 438 may comprise a machine connector base distal end 439 and a machine connector base proximal end 449. In one or more embodiments, machine connector 440 may comprise a retaining ring distal interface 437, a retaining ring proximal interface 447, and a retaining ring 448. Illustratively, retaining ring 448 may be disposed between retaining ring distal interface 437 and retaining ring proximal interface 447. In one or more embodiments, lanyard cable 460 may comprise a lanyard cable distal end 461 and a lanyard cable proximal end 462. Illustratively, machine coupler 450 may comprise a machine coupler inferior end 451 and a machine coupler superior end 452. In one or more embodiments, machine coupler 450 may comprise a machine coupler aperture 453. Illustratively, machine coupler 450 may comprise a machine interface 455.

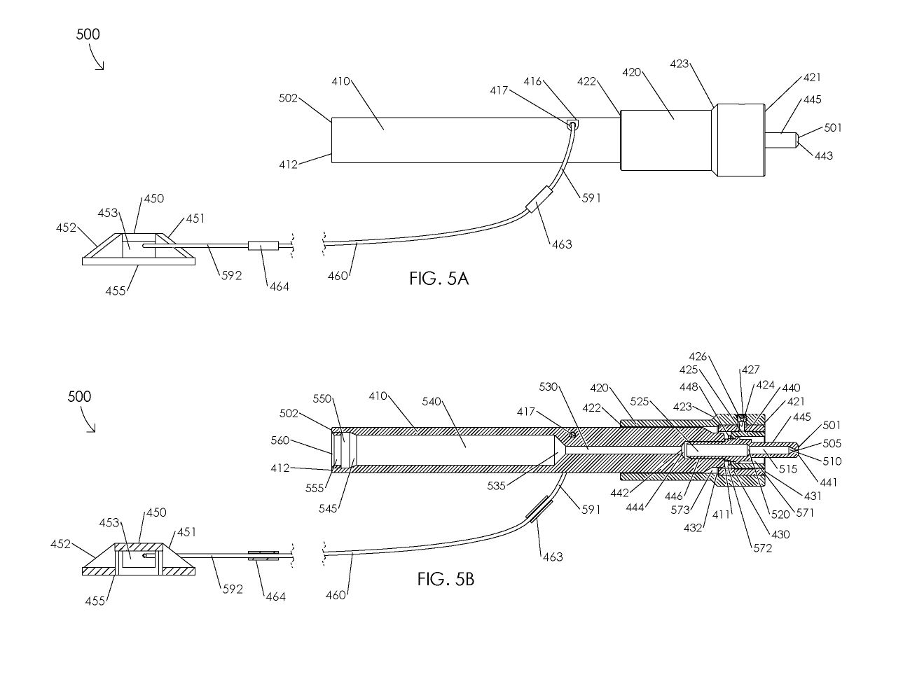

FIGS. 5A and 5B are schematic diagrams illustrating an assembled optic fiber fixture 500. FIG. 5A illustrates a side view of an assembled optic fiber fixture 500. FIG. 5B illustrates a cross-sectional view in a sagittal plane of an assembled optic fiber fixture 500. Illustratively, an assembled optic fiber fixture 500 may comprise an assembled optic fiber fixture distal end 501 and an assembled optic fiber fixture proximal end 502. In one or more embodiments, assembled optic fiber fixture 500 may comprise an optic fiber proximal end guide 505, an inner lumen distal taper 510, a machine connector distal inner lumen 515, an inner lumen proximal taper 520, a machine connector proximal inner lumen 525, a fixture base inner bore 530, a transitory connector proximal housing distal taper 535, a transitory connector proximal housing 540, a transitory connector proximal housing proximal taper 545, a proximal barb interface 550, a proximal arm interface 555, and a fixture base inner taper 560.

In one or more embodiments, lanyard cable 460 may comprise a distal loop 591 and a proximal loop 592. Illustratively, distal fastener 463 may be disposed over a portion of lanyard cable 460 wherein lanyard cable distal end 461 extends a distance from distal fastener 463. In one or more embodiments, lanyard cable distal end 461 may be threaded through lanyard cable guide 417 and into a portion of distal fastener 463 to form distal loop 591. Illustratively, distal fastener 463 may be configured to fix lanyard cable distal end 461 within distal fastener 463, e.g., distal fastener 463 may be configured to fix lanyard cable distal end 461 within distal fastener 463 by an adhesive, a crimp, a weld, a friction fit, etc. In one or more embodiments, proximal fastener 464 may be disposed over a portion of lanyard cable 460 wherein lanyard cable proximal end 462 extends a distance from proximal fastener 464. Illustratively, lanyard cable proximal end 462 may be threaded through machine coupler aperture 453 and into a portion of proximal fastener 464 to form proximal loop 592. In one or more embodiments, proximal fastener 464 may be configured to fix lanyard cable proximal end 462 within proximal fastener 464, e.g., proximal fastener 464 may be configured to fix lanyard cable proximal end 462 within proximal fastener 464 by an adhesive, a crimp, a weld, a friction fit, etc.

Illustratively, machine connector 440 may comprise a machine connector inner chamber 571. In one or more embodiments, distal ferrule 445 may extend a distance out from machine connector inner chamber 571. Illustratively, extender 430 may comprise an extender inner chamber 572. In one or more embodiments, machine connector 440 may be disposed in extender inner chamber 572. Illustratively, machine connector 440 may be disposed in extender 430, e.g., machine connector 440 may be disposed in extender 430 wherein machine connector distal end 441 may extend a distance from extender distal end 431 and wherein machine connector proximal end 442 may extend a distance from extender proximal end 432. In one or more embodiments, machine connector 440 may be fixed in extender 430, e.g., machine connector 440 may be fixed in extender 430 by an adhesive, a crimp, a weld, a friction fit, etc. Illustratively, machine connector housing 420 may comprise a machine connector housing inner chamber 573. In one or more embodiments, extender 430 may be disposed in machine connector housing inner chamber 573. Illustratively, extender 430 may be disposed in machine connector housing 420, e.g., extender 430 may be disposed in machine connector housing 420 wherein extender distal end 431 is disposed between machine connector housing distal end 421 and machine connector housing proximal end 422 and wherein extender proximal end 432 is disposed between machine connector housing distal end 421 and machine connector housing proximal end 422. In one or more embodiments, extender 430 may be disposed in machine connector housing 420 wherein machine connector distal end 441 extends a distance from machine connector housing distal end 421, e.g., extender may be disposed in machine connector housing 420 wherein machine connector proximal end 442 may be disposed between machine connector housing distal end 421 and machine connector housing proximal end 422. Illustratively, extender 430 may be fixed in machine connector housing 420, e.g., extender 430 may be fixed in machine connector housing 420 by an adhesive, a crimp, a weld, a friction fit, etc.

In one or more embodiments, fixture base 410 may be disposed in machine connector housing 420, e.g., fixture base 410 may be disposed in machine connector housing 420 wherein fixture base proximal end 412 extends a distance from machine connector housing proximal end 422 and wherein fixture base distal end 411 is disposed between machine connector housing distal end 421 and machine connector housing proximal end 422. Illustratively, fixture base 410 may be fixed in machine connector housing 420, e.g., fixture base 410 may be fixed in machine connector housing 420 by an adhesive, a crimp, a weld, a friction fit, etc. In one or more embodiments, fixture base 410 may be disposed in extender 430, e.g., fixture base 410 may be disposed in extender 430 wherein fixture base proximal end 412 extends a distance from extender proximal end 432 and wherein fixture base distal end 411 is disposed between extender distal end 431 and extender proximal end 432. Illustratively, fixture base 410 may be fixed in extender 430, e.g., fixture base 410 may be fixed in extender 430 by an adhesive, a crimp, a weld, a friction fit, etc. In one or more embodiments, machine connector 440 may be disposed in fixture base 410, e.g., machine connector 440 may be disposed in fixture base 410 wherein machine connector distal end 441 extends a distance from fixture base distal end 411 and wherein machine connector proximal end 442 is disposed between fixture base distal end 411 and fixture base proximal end 412. Illustratively, machine connector 440 may be fixed in fixture base 410, e.g., machine connector 440 may be fixed in fixture base 410 by an adhesive, a crimp, a weld, a friction fit, etc.

In one or more embodiments, electrical element 424 may be disposed in machine connector housing 420 and extender 430, e.g., electrical element may be disposed in electrical element housing 435. Illustratively, electrical element 424 may be fixed in electrical element housing 435, e.g., electrical element 424 may be fixed in electrical element housing 435 by an adhesive, a crimp, a weld, a friction fit, etc. In one or more embodiments, electrical element 424 may be disposed in machine connector housing 420 and extender 430 wherein electrical element inferior end 425 may be in contact with machine connector 440, e.g., electrical element 424 may be disposed in electrical element housing 435 wherein electrical element inferior end 425 may be in contact with machine connector 440. Illustratively, electrical element 424 may be electrically connected to machine connector 440. In one or more embodiments, electrical element 424 may be configured to convey data to a machine, e.g., electrical element 424 may be configured to convey data to a laser machine. Illustratively, electrical element 424 may comprise a resistor, e.g., electrical element 424 may comprise a cylindrical resistor. In one or more embodiments, electrical element 424 may comprise a radio frequency identification chip.

Illustratively, fixation mechanism 427 may be disposed in machine connector housing 420, e.g., fixation mechanism 427 may be disposed in machine connector housing 420 wherein a portion of fixation mechanism 427 contacts a portion of electrical element 424. In one or more embodiments, fixation mechanism 427 may be fixed in machine connector housing 420, e.g., fixation mechanism 427 may be fixed in machine connector housing 420 by an adhesive, a crimp, a weld, a friction fit, etc. Illustratively, fixation mechanism 427 may be configured to fix electrical element 424 in electrical element housing 435, e.g., fixation mechanism 427 may comprise a setscrew configured to fix electrical element 424 in electrical element housing 435. In one or more embodiments, fixation mechanism 427 may be electrically conductive. Illustratively, fixation mechanism 427 may be disposed in machine connector housing 420 wherein fixation mechanism 427 contacts electrical element 424 and electrical element 424 contacts machine connector 440, e.g., fixation mechanism 427 may be disposed in machine connector housing 420 wherein fixation mechanism 427 contacts electrical element superior end 426 and electrical element inferior end 425 contacts machine connector 440. In one or more embodiments, fixation mechanism 427 may be disposed in machine connector housing 420 wherein fixation mechanism 427 is electrically connected to electrical element 424 and electrical element 424 is electrically connected to machine connector 440.

Illustratively, machine coupler 450 may be configured to attach assembled optic fiber fixture 500 to a laser machine, e.g., machine interface 455 may be configured to attach assembled optic fiber fixture 500 to a laser machine. In one or more embodiments, machine interface 455 may comprise a magnet configured to attach assembled optic fiber fixture 500 to a laser machine. Illustratively, machine interface 455 may comprise an adhesive configured to attach assembled optic fiber fixture 500 to a laser machine. In one or more embodiments, assembled optic fiber fixture 500 may be reusable, e.g., assembled optic fiber fixture 500 may be sold non-sterile and not intended to be sterilized by a user in a medical autoclave. Illustratively, a user may clean assembled optic fiber fixture 500 by flushing assembled optic fiber fixture 500 with a syringe of isopropyl alcohol. In one or more embodiments, flushing assembled optic fiber fixture 500 with a syringe of isopropyl alcohol before each use of assembled optic fiber fixture 500 may be configured to remove any particulate matter that may have accumulated in assembled optic fiber fixture 500 since a previous use of assembled optic fiber fixture 500. Illustratively, optic fiber fixture 500 may comprise an end cap configured to fit over optic fiber fixture proximal end 502, e.g., optic fiber fixture 500 may comprise an end cap configured to fit over optic fiber fixture proximal end 502 to prevent particulate matter from accumulating in optic fiber fixture 500 when optic fiber fixture 500 is not being used by a user.

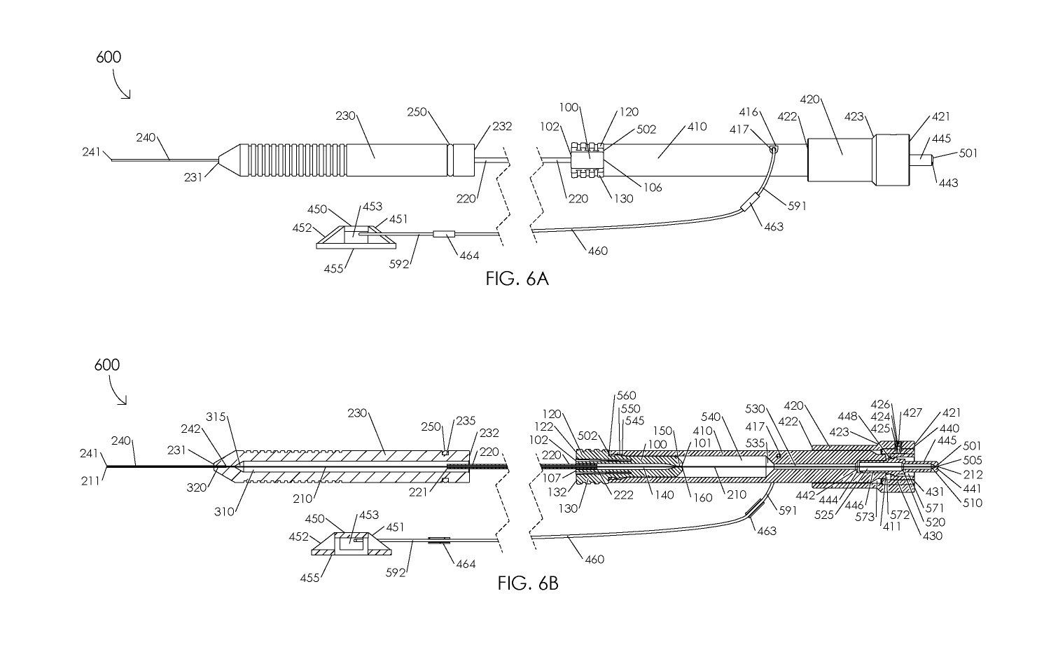

FIGS. 6A and 6B are schematic diagrams illustrating a single-use laser probe with a reusable optic fiber fixture 600. FIG. 6A illustrates a side view of a single-use laser probe with a reusable optic fiber fixture 600. FIG. 6B illustrates a cross-sectional view in a sagittal plane of a single-use laser probe with a reusable optic fiber fixture 600. Illustratively, a single-use laser probe with a reusable optic fiber fixture 600 may comprise an assembled single-use laser probe 300 and an assembled optic fiber fixture 500. In one or more embodiments, a portion of transitory connector 100 may be disposed in assembled optic fiber fixture 500, e.g., transitory connector distal end 101 may be disposed in transitory connector proximal housing 540. Illustratively, a portion of transitory connector 100 may extend a distance from assembled optic fiber fixture proximal end 502, e.g., transitory connector proximal end 102 may extend a distance from assembled optic fiber fixture proximal end 502. In one or more embodiments, a portion of transitory connector 100 may be disposed in assembled optic fiber fixture 500 wherein superior arm barb 123 is disposed in proximal barb interface 550 and inferior arm barb 133 is disposed in proximal barb interface 550. Illustratively, a portion of transitory connector 100 may be disposed in assembled optic fiber fixture 500 wherein superior arm barb 123 is in contact with an outer perimeter of proximal barb interface 550 and wherein inferior arm barb 133 is in contact with the outer perimeter of proximal barb interface 550. In one or more embodiments, a portion of transitory connector 100 may be disposed in assembled optic fiber fixture 500 wherein superior arm 120 and inferior arm 130 are disposed in proximal arm interface 555. Illustratively, a portion of transitory connector 100 may be disposed in assembled optic fiber fixture 500 wherein superior arm 120 is in contact with an outer perimeter of proximal arm interface 555 and wherein inferior arm 130 is in contact with the outer perimeter of proximal arm interface 555. In one or more embodiments, transitory connector proximal housing proximal taper 545 may be configured to prevent superior arm barb 123 and inferior arm barb 133 from advancing into transitory connector proximal housing 540, e.g., a portion of superior arm barb 123 and a portion of inferior arm barb 133 may contact an outer perimeter of transitory connector proximal housing proximal taper 545.

Illustratively, inserting a portion of transitory connector 100 into transitory connector proximal housing 540 may be configured to ingress optic fiber proximal end 212 into transitory connector proximal housing distal taper 535. In one or more embodiments, transitory connector proximal housing distal taper 535 may be configured to guide an ingress of optic fiber proximal end 212 into fixture base inner bore 530. Illustratively, fixture base inner bore 530 may be configured to guide an ingress of optic fiber proximal end 212 into machine connector proximal inner lumen 525. In one or more embodiments, inner lumen proximal taper 520 may be configured to guide an ingress of optic fiber proximal end 212 into machine connector distal inner lumen 515. Illustratively, inner lumen distal taper 510 may be configured to guide an ingress of optic fiber proximal end 212 into optic fiber proximal end guide 505. In one or more embodiments, a distance that optic fiber proximal end 212 extends from transitory connector distal end 101 may be configured to cause optic fiber proximal end 212 to be adjacent to assembled optic fiber fixture proximal end 502 when transitory connector 100 is inserted into transitory connector proximal housing 540. Illustratively, a distance that optic fiber proximal end 212 extends from transitory connector distal end 101 may be configured to cause optic fiber proximal end 212 to be coplanar with assembled optic fiber fixture proximal end 502 when transitory connector 100 is inserted into transitory connector proximal housing 540.

Illustratively, a user may perform a photocoagulation procedure with a single-use laser probe with a reusable optic fiber fixture 600. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site. In one or more embodiments, assembled single-use laser probe 300 may comprise a single-use, disposable medical device. Illustratively, assembled single-use laser probe 300 may be packaged in a sealed pouch and sterilized by ethylene oxide. In one or more embodiments, assembled optic fiber fixture 500 may comprise a reusable medical device accessory. Illustratively, assembled optic fiber fixture 500 may be sold non-sterile and used non-sterile.

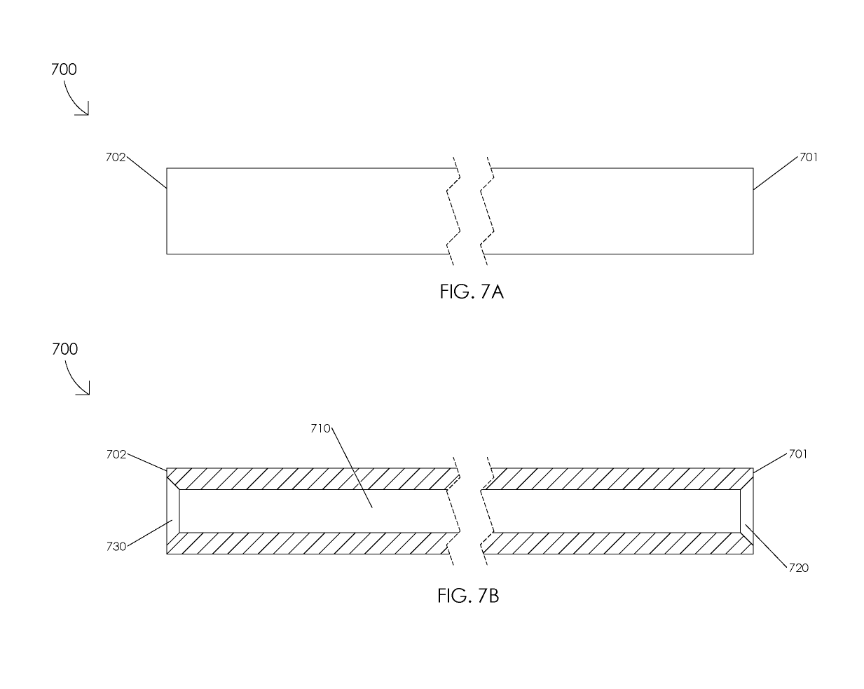

FIGS. 7A and 7B are schematic diagrams illustrating a one-piece handle 700. FIG. 7A illustrates a side view of a one-piece handle 700. Illustratively, one-piece handle 700 may comprise a one-piece handle distal end 701 and a one-piece handle proximal end 702. FIG. 7B illustrates a cross-sectional view in a sagittal plane of a one-piece handle 700. In one or more embodiments, a one-piece handle 700 may comprise a one-piece handle inner bore 710, a one-piece handle inner bore distal taper 720, and a one-piece handle inner bore proximal taper 730. Illustratively, a portion of optic fiber 210 may be disposed in one-piece handle 700, e.g., optic fiber 210 may be disposed in one-piece handle 700 wherein optic fiber distal end 211 is adjacent to one-piece handle distal end 710. In one or more embodiments, optic fiber 210 may be disposed in one-piece handle inner bore proximal taper 730, one-piece handle inner bore 710, and one-piece handle inner bore distal taper 720. Illustratively, optic fiber 210 may be disposed in one-piece handle 700 wherein optic fiber distal end 211 is coplanar with one-piece handle distal end 701. In one or more embodiments, one-piece handle inner bore proximal taper 730 may be configured to guide an ingress of optic fiber distal end 211 into one-piece handle inner bore 710.

Illustratively, one-piece handle inner bore distal taper 720 may be configured to prevent degradation to a laser spot wherein optic fiber distal end 211 is recessed from one-piece handle distal end 701 relative to a laser spot wherein optic fiber distal end 211 is coplanar with one-piece handle distal end 701. In one or more embodiments, one-piece handle inner bore distal taper 720 may be configured to ensure that a power output wherein optic fiber distal end 211 is recessed relative to one-piece handle distal end 701 is identical to a power output wherein optic fiber distal end 211 is coplanar with one-piece handle distal end 701. Illustratively, one-piece handle inner bore distal taper 720 may be configured to ensure that a laser spot size wherein optic fiber distal end 211 is recessed relative to one-piece handle distal end 701 is identical to a laser spot size wherein optic fiber distal end 211 is coplanar with one-piece handle distal end 701. In one or more embodiments, one-piece handle inner bore distal taper 720 may be configured to ensure that a laser spot shape wherein optic fiber distal end 211 is recessed relative to one-piece handle distal end 701 is identical to a laser spot shape wherein optic fiber distal end 211 is coplanar with one-piece handle distal end 701. Illustratively, optic fiber 210 may be fixed within one-piece handle 700, e.g., optic fiber 210 may be fixed within one-piece handle 700 by a friction fit, an adhesive, a crimp, a weld, etc.

Illustratively, optic fiber 210 may be disposed in jacketing 220, e.g., optic fiber 210 may be disposed in jacketing 220 wherein one or more portions of optic fiber 210 extend from one or more portions of jacketing 220. In one or more embodiments, optic fiber 210 may be disposed in jacketing 220 wherein optic fiber distal end 211 extends a distance from jacketing distal end 221. Illustratively, optic fiber 210 may be disposed in jacketing 220 wherein optic fiber proximal end 212 extends a distance from jacketing proximal end 222. In one or more embodiments, a portion of jacketing 220 may be disposed in a portion of transitory connector 100, e.g., jacketing proximal end 222 may be disposed in a portion of transitory connector 100. Illustratively, jacketing proximal end 222 may be disposed in tapered inner lumen 140. In one or more embodiments, a portion of jacketing 220 may be fixed within a portion of transitory connector 100, e.g., a portion of jacketing 220 may be fixed within a portion of transitory connector 100 by a friction fit, an adhesive, a crimp, a weld, etc. Illustratively, a portion of one-piece handle 700 may be disposed in a portion of jacketing 220, e.g., one-piece handle proximal end 702 may be disposed in a portion of jacketing 220. In one or more embodiments, one-piece handle proximal end 702 may be disposed in jacketing distal end 221. Illustratively, a portion of one-piece handle 700 may be fixed within a portion of jacketing 220, e.g., a portion of one-piece handle 700 may be fixed within a portion of jacketing 220 by a friction fit, an adhesive, a crimp, a weld, etc.

In one or more embodiments, a portion of optic fiber 210 may be disposed in transitory connector 100, e.g., optic fiber 210 may be disposed in tapered inner lumen 140, optic fiber housing 150, and fixation mechanism housing 160. Illustratively, optic fiber 210 may be fixed in a position relative to transitory connector 100, e.g., optic fiber 210 may be fixed within transitory connector 100 wherein optic fiber proximal end 212 extends a distance from transitory connector distal end 101. In one or more embodiments, a portion of optic fiber 210 may be fixed within a portion of transitory connector 100, e.g., a portion of optic fiber 210 may be fixed within a portion of transitory connector 100 by a friction fit, an adhesive, a crimp, a weld, etc. Illustratively, a fixation mechanism may be configured to fix a portion of optic fiber 210 within optic fiber housing 150, e.g., a fixation mechanism may be disposed in fixation mechanism housing 160 wherein the fixation mechanism is configured to fix a portion of optic fiber 210 within optic fiber housing 150.

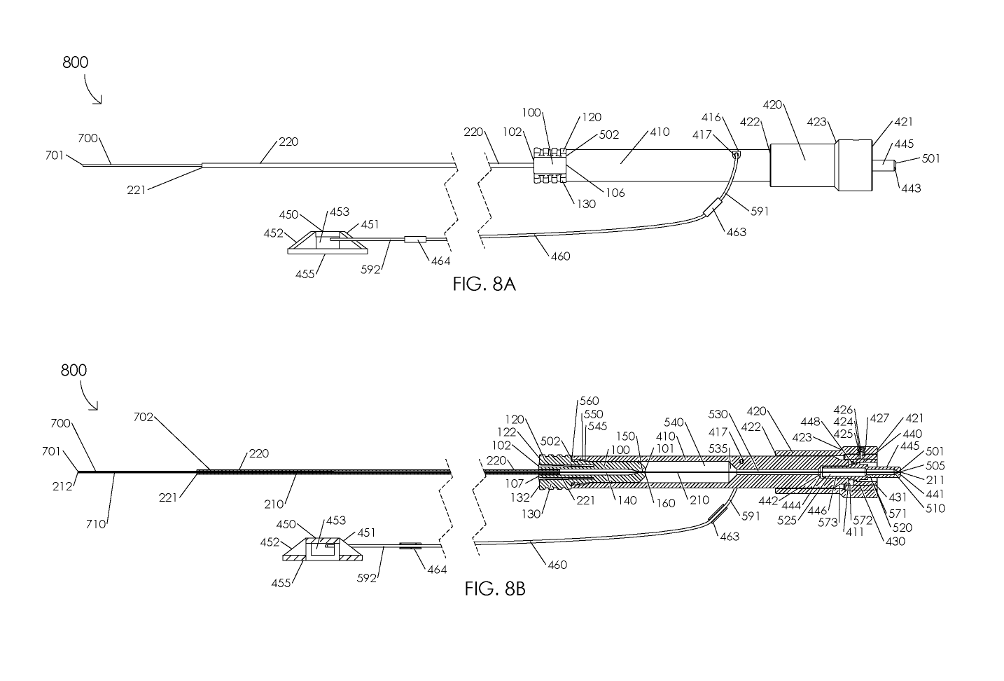

FIGS. 8A and 8B are schematic diagrams illustrating a single-use one-piece laser probe with a reusable optic fiber fixture 800. FIG. 8A illustrates a side view of a single-use one-piece laser probe with a reusable optic fiber fixture 800. FIG. 8B illustrates a cross-sectional view in a sagittal plane of a single-use one-piece laser probe with a reusable optic fiber fixture 800. Illustratively, a single-use one-piece laser probe with a reusable optic fiber fixture 800 may comprise a one-piece handle 700, an optic fiber 210, a jacketing 220, a transitory connector 100, and an assembled optic fiber fixture 500. In one or more embodiments, a portion of transitory connector 100 may be disposed in assembled optic fiber fixture 500, e.g., transitory connector distal end 101 may be disposed in transitory connector proximal housing 540. Illustratively, a portion of transitory connector 100 may extend a distance from assembled optic fiber fixture proximal end 502, e.g., transitory connector proximal end 102 may extend a distance from assembled optic fiber fixture proximal end 502. In one or more embodiments, a portion of transitory connector 100 may be disposed in assembled optic fiber fixture 500 wherein superior arm barb 123 is disposed in proximal barb interface 550 and inferior arm barb 133 is disposed in proximal barb interface 550. Illustratively, a portion of transitory connector 100 may be disposed in assembled optic fiber fixture 500 wherein superior arm barb 123 is in contact with an outer perimeter of proximal barb interface 550 and wherein inferior arm barb 133 is in contact with the outer perimeter of proximal barb interface 550. In one or more embodiments, a portion of transitory connector 100 may be disposed in assembled optic fiber fixture 500 wherein superior arm 120 and inferior arm 130 are disposed in proximal arm interface 555. Illustratively, a portion of transitory connector 100 may be disposed in assembled optic fiber fixture 500 wherein superior arm 120 is in contact with an outer perimeter of proximal arm interface 555 and wherein inferior arm 130 is in contact with the outer perimeter of proximal arm interface 555. In one or more embodiments, transitory connector proximal housing proximal taper 545 may be configured to prevent superior arm barb 123 and inferior arm barb 133 from advancing into transitory connector proximal housing 540, e.g., a portion of superior arm barb 123 and a portion of inferior arm barb 133 may contact an outer perimeter of transitory connector proximal housing proximal taper 545.

is Illustratively, inserting a portion of transitory connector 100 into transitory connector proximal housing 540 may be configured to ingress optic fiber proximal end 212 into transitory connector proximal housing distal taper 535. In one or more embodiments, transitory connector proximal housing distal taper 535 may be configured to guide an ingress of optic fiber proximal end 212 into fixture base inner bore 530. Illustratively, fixture base inner bore 530 may be configured to guide an ingress of optic fiber proximal end 212 into machine connector proximal inner lumen 525. In one or more embodiments, inner lumen proximal taper 520 may be configured to guide an ingress of optic fiber proximal end 212 into machine connector distal inner lumen 515. Illustratively, inner lumen distal taper 510 may be configured to guide an ingress of optic fiber proximal end 212 into optic fiber proximal end guide 505. In one or more embodiments, a distance that optic fiber proximal end 212 extends from transitory connector distal end 101 may be configured to cause optic fiber proximal end 212 to be adjacent to assembled optic fiber fixture proximal end 502 when transitory connector 100 is inserted into transitory connector proximal housing 540.

Illustratively, a user may perform a photocoagulation procedure with a single-use one-piece laser probe with a reusable optic fiber fixture 800. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site. In one or more embodiments, assembled optic fiber fixture 500 may comprise a reusable medical device accessory. Illustratively, assembled optic fiber fixture 500 may be sold non-sterile and used non-sterile. In one or more embodiments, one-piece handle 700, optic fiber 210, jacketing 220, and transitory connector 100 may comprise a single-use disposable medical device. Illustratively, one-piece handle 700, optic fiber 210, jacketing 220, and transitory connector 100 may be pouched and sterilized by ethylene oxide.

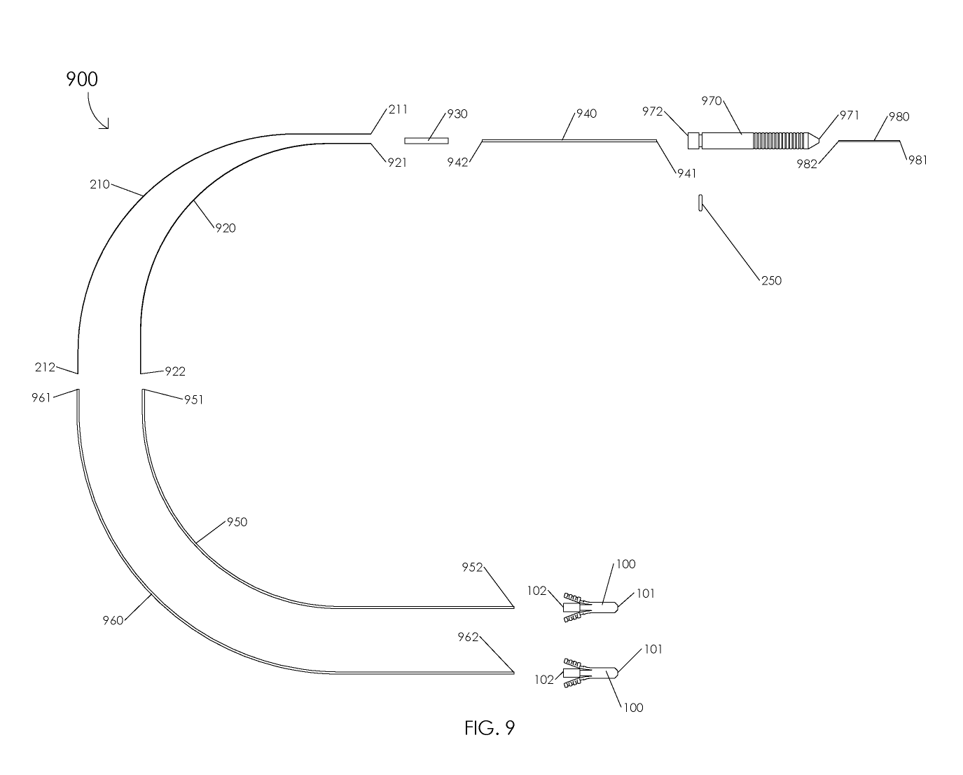

FIG. 9 is a schematic diagram illustrating an exploded view of a single-use illuminated laser probe assembly 900. Illustratively, a single-use illuminated laser probe assembly 900 may comprise a first transitory connector 100, a second transitory connector 100, an optic fiber 210, an illumination optic fiber 920, a coupling sleeve 930, a dual fiber housing 940, an illumination jacketing 950, a jacketing 960, an illumination handle base 970, and an illumination hypodermic tube 980. In one or more embodiments, illumination optic fiber 920 may comprise an illumination optic fiber distal end 921 and an illumination optic fiber proximal end 922. Illustratively, dual fiber housing 940 may comprise a dual fiber housing distal end 941 and a dual fiber housing proximal end 942. In one or more embodiments, illumination jacketing 950 may comprise an illumination jacketing distal end 951 and an illumination jacketing proximal end 952. Illustratively, jacketing 960 may comprise a jacketing distal end 961 and a jacketing proximal end 962. In one or more embodiments, illumination handle base 970 may comprise an illumination handle base distal end 971 and an illumination handle base proximal end 972. Illustratively, illumination hypodermic tube 980 may comprise an illumination hypodermic tube distal end 981 and an illumination hypodermic tube proximal end 982.

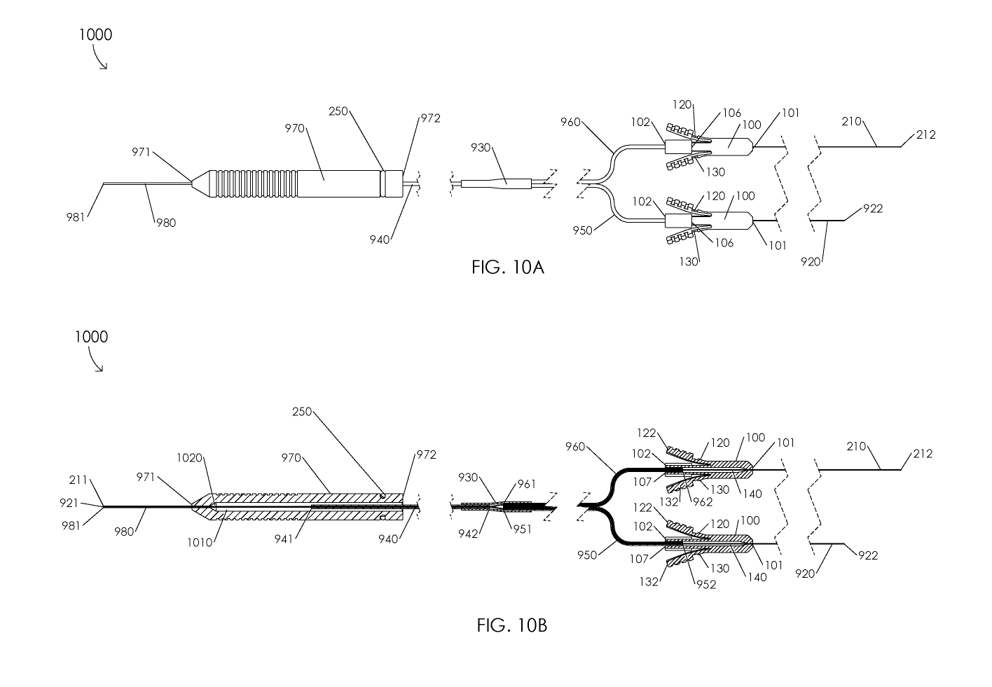

FIGS. 10A and 10B are schematic diagrams illustrating an assembled single-use illuminated laser probe 1000. FIG. 10A illustrates a side view of an assembled single-use illuminated laser probe 1000. FIG. 10B illustrates a cross-sectional view in a sagittal plane of an assembled single-use illuminated laser probe 1000. Illustratively, an assembled single-use illuminated laser probe 1000 may comprise an illumination handle base inner lumen 1010 and an illumination handle base inner lumen distal taper 1020. In one or more embodiments, optic fiber proximal end 212 may extend a distance from first transitory connector distal end 101. Illustratively, illumination fiber proximal end 922 may extend a distance from second transitory connector distal end 101. In one or more embodiments, optic fiber 210 may be disposed in jacketing 960 wherein optic fiber distal end 211 extends a distance from jacketing distal end 961 and wherein optic fiber proximal end 212 extends a distance from jacketing distal end 962. Illustratively, illumination optic fiber 920 may be disposed in illumination jacketing 950 wherein illumination optic fiber distal end 921 extends a distance from illumination jacketing distal end 951 and wherein illumination optic fiber proximal end 922 extends a distance from illumination jacketing proximal end 952.

In one or more embodiments, a portion of illumination hypodermic tube 980 may be disposed in illumination handle base 970, e.g., illumination hypodermic tube proximal end 982 may be disposed in illumination handle base 970. Illustratively, illumination hypodermic tube proximal end 982 may be fixed in illumination handle base 970, e.g., illumination hypodermic tube proximal end 982 may be fixed in illumination handle base 970 by a friction fit, an adhesive, a weld, a setscrew, etc. In one or more embodiments, a portion of illumination jacketing 950 may be disposed in coupling sleeve 930, e.g., illumination jacketing distal end 951 may be disposed in coupling sleeve 930. Illustratively, illumination jacketing distal end 951 may be fixed in coupling sleeve 930. In one or more embodiments, a portion of jacketing 960 may be disposed in coupling sleeve 930, e.g., jacketing distal end 961 may be disposed in coupling sleeve 930. Illustratively, jacketing distal end 961 may be fixed in coupling sleeve 930. In one or more embodiments, a portion of dual fiber housing 940 may be disposed in coupling sleeve 930, e.g., dual fiber housing proximal end 942 may be disposed in coupling sleeve 930. Illustratively, dual fiber housing proximal end 942 may be fixed in coupling sleeve 930. In one or more embodiments, a portion of dual fiber housing 940 may be disposed in illumination handle base 970, e.g., dual fiber housing distal end 941 may be disposed in illumination handle base inner lumen 1010. Illustratively, dual fiber housing distal end 941 may be fixed in illumination handle base inner lumen 1010, e.g., dual fiber housing distal end 941 may be fixed in illumination handle base inner lumen 1010 by a friction fit, an adhesive, a weld, a setscrew, etc.

In one or more embodiments, optic fiber 210 may be disposed in coupling sleeve 930, dual fiber housing 940, illumination handle base inner lumen 1010, illumination handle base inner lumen distal taper 1020, and illumination hypodermic tube 980. Illustratively, optic fiber 210 may be disposed in illumination hypodermic tube 980 wherein optic fiber distal end 211 is adjacent to illumination hypodermic tube distal end 981, e.g., optic fiber 210 may be disposed in illumination hypodermic tube 980 wherein optic fiber distal end 211 is coplanar with illumination hypodermic tube distal end 981. In one or more embodiments, optic fiber 210 may be fixed in hypodermic tube 980, e.g., optic fiber 210 may be fixed in hypodermic tube 980 by an adhesive, an epoxy, or any suitable fixation means. Illustratively, illumination optic fiber 920 may be disposed in coupling sleeve 930, dual fiber housing 940, illumination handle base inner lumen 1010, illumination handle base inner lumen distal taper 1020, and illumination hypodermic tube 980. In one or more embodiments, illumination optic fiber 920 may be disposed in illumination hypodermic tube 980 wherein illumination optic fiber distal end 921 is adjacent to illumination hypodermic tube distal end 981, e.g., illumination optic fiber 920 may be disposed in illumination hypodermic tube 980 wherein illumination optic fiber distal end 920 is coplanar with illumination hypodermic tube distal end 981. Illustratively, illumination optic fiber 920 may be fixed in hypodermic tube 980, e.g., illumination optic fiber 920 may be fixed in hypodermic tube 980 by an adhesive, an epoxy, or any suitable fixation means.

In one or more embodiments, a portion of jacketing 960 may be disposed in a portion of first transitory connector 100, e.g., jacketing proximal end 962 may be disposed in tapered inner lumen 140. Illustratively, a portion of jacketing 960 may be fixed in a portion of first transitory connector 100, e.g., a portion of jacketing 960 may be fixed in a portion of first transitory connector 100 by an adhesive, a friction fit, a crimp, a tie, a weld, etc. In one or more embodiments, a portion of illumination jacketing 950 may be disposed in a portion of second transitory connector 100, e.g., illumination jacketing proximal end 952 may be disposed in tapered inner lumen 140. Illustratively, a portion of illumination jacketing 950 may be fixed in a portion of second transitory connector 100, e.g., a portion of illumination jacketing 950 may be fixed in a portion of second transitory connector 100 by an adhesive, a friction fit, a crimp, a tie, a weld, etc.

In one or more embodiments, optic fiber 210 may be disposed in first transitory connector 100 wherein optic fiber 210 is disposed in tapered inner lumen 140, optic fiber housing 150, and fixation mechanism housing 160. Illustratively, a portion of optic fiber 210 may be fixed within fixation mechanism housing 160, e.g., a portion of optic fiber 210 may be fixed within fixation mechanism housing 160 by an adhesive, a friction fit, a crimp, a tie, a weld, etc. In one or more embodiments, optic fiber 210 may be fixed in first transitory connector 100 wherein optic fiber proximal end 212 extends a fixed distance from first transitory connector distal end 101. Illustratively, illumination optic fiber 920 may be disposed in second transitory connector 100 wherein illumination optic fiber 920 is disposed in tapered inner lumen 140, optic fiber housing 150, and fixation mechanism housing 160. Illustratively, a portion of illumination optic fiber 920 may be fixed within fixation mechanism housing 160, e.g., a portion of illumination optic fiber 920 may be fixed within fixation mechanism housing 160 by an adhesive, a friction fit, a crimp, a tie, a weld, etc. In one or more embodiments, illumination optic fiber 920 may be fixed in second transitory connector 100 wherein illumination optic fiber proximal end 922 extends a fixed distance from second transitory connector distal end 101. Illustratively, a distance that optic fiber proximal end 212 extends from first transitory connector distal end 101 may be different from a distance that illumination optic fiber proximal end 922 extends from second transitory connector distal end 101. In one or more embodiments, a distance that optic fiber proximal end 212 extends from first transitory connector distal end 101 and a distance that illumination optic fiber proximal end 922 extends from second transitory connector distal end 101 may be configured to prevent a user from inserting optic fiber 210 into an illumination machine and to prevent a user from inserting illumination optic fiber 920 into a laser machine.

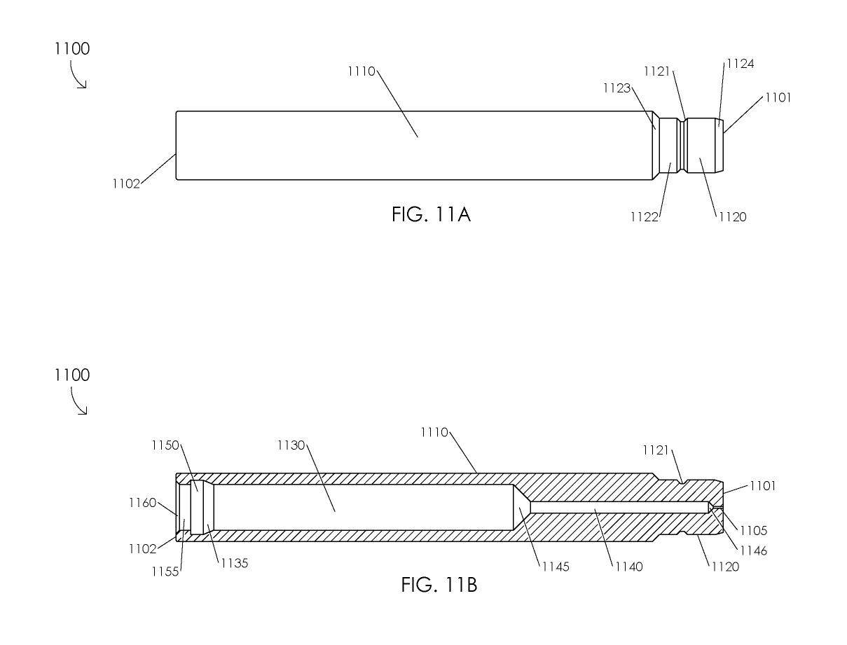

FIGS. 11A and 11B are schematic diagrams illustrating an illumination optic fiber fixture 1100. FIG. 11A illustrates a side view of an illumination optic fiber fixture 1100. Illustratively, an illumination optic fiber fixture 1100 may comprise an illumination optic fiber fixture distal end 1101 and an illumination optic fiber fixture proximal end 1102. In one or more embodiments, an illumination optic fiber fixture 1100 may comprise an illumination fixture base 1110, an illumination machine connector 1120, a channel 1121, an illumination machine connector base 1122, an illumination machine connector proximal taper 1123, and an illumination machine connector distal taper 1124. FIG. 11B illustrates a cross-sectional view in a sagittal plane of an illumination optic fiber fixture 1100. Illustratively, illumination optic fiber fixture 1100 may comprise an illumination optic fiber guide 1105, a transitory connector proximal housing 1130, a transitory connector proximal housing proximal taper 1135, an illumination optic fiber fixture inner bore 1140, a transitory connector proximal housing distal taper 1145, an illumination optic fiber fixture inner bore distal taper 1146, a proximal barb interface 1150, a proximal arm interface 1155, and an illumination optic fiber fixture inner taper 1160.

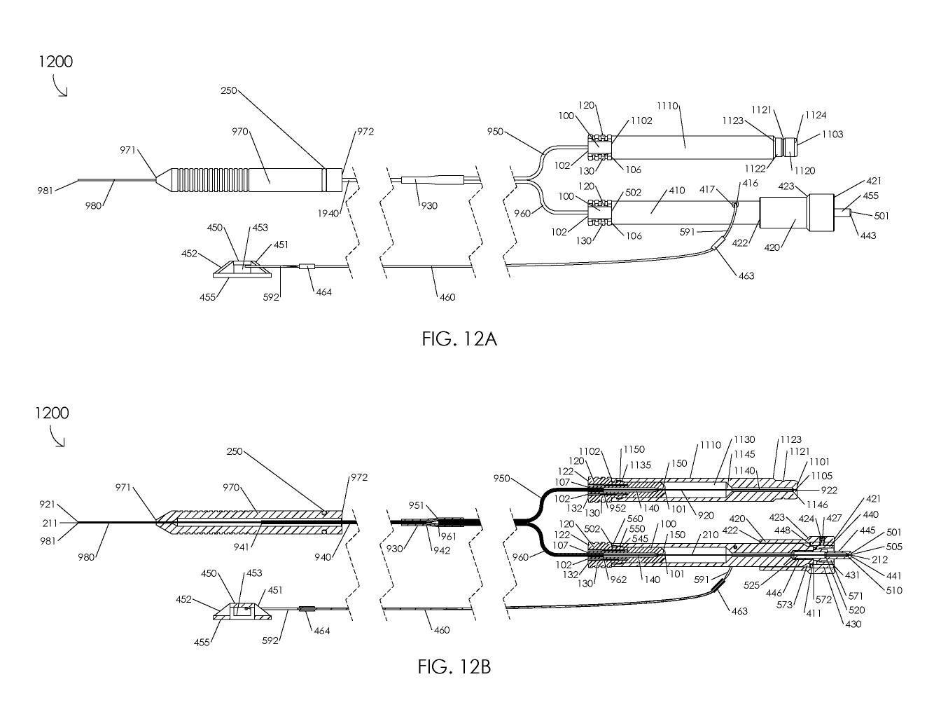

FIGS. 12A and 12B are schematic diagrams illustrating a single-use illuminated laser probe with a reusable optic fiber fixture and a reusable illumination optic fiber fixture 1200. FIG. 12A illustrates a side view of a single-use illuminated laser probe with a reusable optic fiber fixture and a reusable illumination optic fiber fixture 1200. FIG. 12B illustrates a cross-sectional view in a sagittal plane of a single-use illuminated laser probe with a reusable optic fiber fixture and a reusable illumination optic fiber fixture 1200. Illustratively, a single-use illuminated laser probe with a reusable optic fiber fixture and a reusable illumination optic fiber fixture 1200 may comprise an assembled single-use illuminated laser probe, an illumination optic fiber fixture 1100, and an assembled optic fiber fixture 500. In one or more embodiments, a portion of a first transitory connector 100 may be disposed in assembled optic fiber fixture 500, e.g., first transitory connector distal end 101 may be disposed in transitory connector proximal housing 540. Illustratively, a portion of first transitory connector 100 may extend a distance from assembled optic fiber fixture proximal end 502, e.g., first transitory connector proximal end 102 may extend a distance from assembled optic fiber fixture proximal end 502. In one or more embodiments, a portion of first transitory connector 100 may be disposed in assembled optic fiber fixture 500 wherein superior arm barb 123 is disposed in proximal barb interface 550 and inferior arm barb 133 is disposed in proximal barb interface 550. Illustratively, a portion of first transitory connector 100 may be disposed in assembled optic fiber fixture 500 wherein superior arm barb 123 is in contact with an outer perimeter of proximal barb interface 550 and wherein inferior arm barb 133 is in contact with the outer perimeter of proximal barb interface 550. In one or more embodiments, a portion of first transitory connector 100 may be disposed in assembled optic fiber fixture 500 wherein superior arm 120 and inferior arm 130 are disposed in proximal arm interface 555. Illustratively, a portion of first transitory connector 100 may be disposed in assembled optic fiber fixture 500 wherein superior arm 120 is in contact with an outer perimeter of proximal arm interface 555 and wherein inferior arm 130 is in contact with the outer perimeter of proximal arm interface 555. In one or more embodiments, transitory connector proximal housing proximal taper 545 may be configured to prevent superior arm barb 123 and inferior arm barb 133 from advancing into transitory connector proximal housing 540, e.g., a portion of superior arm barb 123 and a portion of inferior arm barb 133 may contact an outer perimeter of transitory connector proximal housing proximal taper 545.

Illustratively, inserting a portion of first transitory connector 100 into transitory connector proximal housing 540 may be configured to ingress optic fiber proximal end 212 into transitory connector proximal housing distal taper 535. In one or more embodiments, transitory connector proximal housing distal taper 535 may be configured to guide an ingress of optic fiber proximal end 212 into fixture base inner bore 530. Illustratively, fixture base inner bore 530 may be configured to guide an ingress of optic fiber proximal end 212 into machine connector proximal inner lumen 525. In one or more embodiments, inner lumen proximal taper 520 may be configured to guide an ingress of optic fiber proximal end 212 into machine connector distal inner lumen 515. Illustratively, inner lumen distal taper 510 may be configured to guide an ingress of optic fiber proximal end 212 into optic fiber proximal end guide 505. In one or more embodiments, a distance that optic fiber proximal end 212 extends from first transitory connector distal end 101 may be configured to cause optic fiber proximal end 212 to be adjacent to assembled optic fiber fixture proximal end 502 when first transitory connector 100 is inserted into transitory connector proximal housing 540.

In one or more embodiments, a portion of a second transitory connector 100 may be disposed in illumination optic fiber fixture 1100, e.g., second transitory connector distal end 101 may be disposed in transitory connector proximal housing 1130. Illustratively, a portion of second transitory connector 100 may extend a distance from illumination optic fiber fixture proximal end 1102, e.g., second transitory connector proximal end 102 may extend a distance from illumination optic fiber fixture proximal end 1102. In one or more embodiments, a portion of second transitory connector 100 may be disposed in illumination optic fiber fixture 1100 wherein superior arm barb 123 is disposed in proximal barb interface 1150 and inferior arm barb 133 is disposed in proximal barb interface 1150. Illustratively, a portion of second transitory connector 100 may be disposed in illumination optic fiber fixture 1100 wherein superior arm barb 123 is in contact with an outer perimeter of proximal barb interface 1150 and wherein inferior arm barb 133 is in contact with the outer perimeter of proximal barb interface 1150. In one or more embodiments, a portion of second transitory connector 100 may be disposed in illumination optic fiber fixture 1100 wherein superior arm 120 and inferior arm 130 are disposed in proximal arm interface 1155. Illustratively, a portion of second transitory connector 100 may be disposed in illumination optic fiber fixture 1100 wherein superior arm 120 is in contact with an outer perimeter of proximal arm interface 1155 and wherein inferior arm 130 is in contact with the outer perimeter of proximal arm interface 1155. In one or more embodiments, transitory connector proximal housing proximal taper 1135 may be configured to prevent superior arm barb 123 and inferior arm barb 133 from advancing into transitory connector proximal housing 1130, e.g., a portion of superior arm barb 123 and a portion of inferior arm barb 133 may contact an outer perimeter of transitory connector proximal housing proximal taper 1135.

Illustratively, inserting a portion of second transitory connector 100 into transitory connector proximal housing 1130 may be configured to ingress illumination optic fiber proximal end 922 into transitory connector proximal housing distal taper 1145. In one or more embodiments, transitory connector proximal housing distal taper 1145 may be configured to guide an ingress of illumination optic fiber proximal end 922 into illumination optic fiber fixture inner bore 1140. Illustratively, illumination optic fiber fixture inner bore 1140 may be configured to guide an ingress of illumination optic fiber proximal end 922 into illumination optic fiber fixture inner bore distal taper 1146. In one or more embodiments, illumination optic fiber fixture inner bore distal taper 1146 may be configured to guide an ingress of illumination optic fiber proximal end 922 into illumination optic fiber guide 1105. Illustratively, a distance that illumination optic fiber proximal end 922 extends from second transitory connector distal end 101 may be configured to cause illumination optic fiber proximal end 922 to be adjacent to illumination optic fiber fixture distal end 1101 when second transitory connector 100 is inserted into transitory connector proximal housing 1130.

Illustratively, a user may perform an illuminated photocoagulation procedure with a single-use illuminated laser probe with a reusable optic fiber fixture and a reusable illumination optic fiber fixture 1200. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site. In one or more embodiments, a user may connect illumination machine connector 1120 to an illumination machine. Illustratively, a user may energize the illumination machine to deliver illumination light into illumination optic fiber proximal end 922, through illumination optic fiber 920, out from illumination optic fiber distal end 921, and onto a surgical target site. In one or more embodiments, illumination optic fiber fixture 1100 may be a reusable medical device sold non-sterile and sterilized by a user in a medical autoclave. Illustratively, assembled single-use illuminated laser probe 1000 may be a single-use medical device sold sterile and discarded after use. In one or more embodiments, optic fiber 210 may be manufactured from glass, e.g., optic fiber 210 may be manufactured from silica. Illustratively, optic fiber 210 may comprise a plurality of optic fibers 210. In one or more embodiments, illumination optic fiber 920 may comprise a plurality of illumination optic fibers 920. Illustratively, illumination optic fiber 920 may comprise one or more optic fibers manufactured from plastic, e.g., illumination optic fiber 920 may comprise one or more optic fibers manufactured from Polymethyl Methacrylate Resin, Polystyrene, etc. In one or more embodiments, illumination optic fiber 920 may comprise one or more optic fibers having a cladding material, e.g., illumination optic fiber 920 may comprise one or more optic fibers having a cladding material manufactured from a fluorinated polymer, a silicone resin, etc. Illustratively, illumination optic fiber 920 may comprise one or more optic fibers having a step index refractive index profile. In one or more embodiments, illumination optic fiber 920 may comprise one or more multi-mode optic fibers, one or more single-mode optic fibers, etc. In one or more embodiments, illumination optic fiber 920 may comprise one or more optic fibers having a core refractive index in a range of 1.3 to 1.8, e.g., illumination optic fiber 920 may comprise one or more optic fibers having a core refractive index of 1.49. Illustratively, illumination optic fiber 920 may comprise one or more optic fibers having a core refractive index of less than 1.3 or greater than 1.8. In one or more embodiments, illumination optic fiber 920 may comprise one or more optic fibers having a numerical aperture in a range of 0.3 to 0.8, e.g., illumination optic fiber 920 may comprise one or more optic fibers having a numerical aperture of 0.5. In one or more embodiments, illumination optic fiber 920 may comprise one or more optic fibers having a numerical aperture of less than 0.3 or greater than 0.8. Illustratively, illumination optic fiber 920 may comprise one or more optic fibers having a core diameter in a range of 85 to 285 micrometers, e.g., illumination optic fiber 920 may comprise one or more optic fibers having a core diameter of 135 micrometers. In one or more embodiments, illumination optic fiber 920 may comprise one or more optic fibers having a core diameter of less than 85 micrometers or greater than 285 micrometers. Illustratively, illumination optic fiber 920 may comprise one or more optic fibers having an overall diameter in a range of 100 to 300 micrometers, e.g., illumination optic fiber 920 may comprise one or more optic fiber having an overall diameter of 200 micrometers. In one or more embodiments, illumination optic fiber 920 may comprise one or more optic fibers having an overall diameter of less than 100 or greater than 300 micrometers.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a laser probe, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a first transitory connector having a first transitory connector distal end and a first transitory connector proximal end;
a superior arm of the first transitory connector having a superior arm barb;
an inferior arm of the first transitory connector having an inferior arm barb;
a hypodermic tube having a hypodermic tube distal end and a hypodermic tube proximal end; and
an optic fiber having an optic fiber distal end and an optic fiber proximal end, the optic fiber disposed in the first transitory connector and the hypodermic tube wherein the optic fiber is fixed within the hypodermic tube and the optic fiber distal end is coplanar with the hypodermic tube distal end and wherein the optic fiber is fixed within the first transitory connector and the optic fiber proximal end extends a distance from the first transitory connector distal end.

2. The instrument of claim 1 wherein the first transitory connector is manufactured from a material configured to deform if the material is sterilized in a medical autoclave.

3. The instrument of claim 1 wherein the optic fiber is fixed within an optic fiber housing of the first transitory connector.

4. The instrument of claim 1 wherein the optic fiber is configured to transmit laser light.

5. The instrument of claim 1 wherein the first transitory connector has a tapered inner lumen.

6. The instrument of claim 1 further comprising:
a second transitory connector having a second transitory connector distal end and a second transitory connector proximal end;
a superior arm of the second transitory connector having a superior arm barb; and
an inferior arm of the second transitory connector having an inferior arm barb.

7. The instrument of claim 6 further comprising:
an illumination optic fiber having an illumination optic fiber distal end and an illumination optic fiber proximal end.

8. The instrument of claim 7 wherein the illumination optic fiber is disposed in the second transitory connector.

9. The instrument of claim 8 wherein the illumination optic fiber is fixed within the second transitory connector.

10. The instrument of claim 9 wherein the illumination optic fiber proximal end extends a distance from the second transitory connector distal end.

11. The instrument of claim 10 wherein the illumination optic fiber is disposed within the hypodermic tube.

12. The instrument of claim 11 wherein the illumination optic fiber is fixed within the hypodermic tube.

13. The instrument of claim 12 wherein the illumination optic fiber distal end is coplanar with the hypodermic tube distal end.

14. An instrument comprising:
a first transitory connector having a first transitory connector distal end and a first transitory connector proximal end;
a superior arm of the first transitory connector having a superior arm barb;
an inferior arm of the first transitory connector having an inferior arm barb;
a one-piece handle having a one-piece handle distal end and a one-piece handle proximal end; and
an optic fiber having an optic fiber distal end and an optic fiber proximal end, the optic fiber disposed in the first transitory connector and the one-piece handle wherein the optic fiber is fixed within the one-piece handle and the optic fiber distal end is coplanar with the one-piece handle distal end and wherein the optic fiber is fixed within the first transitory connector and the optic fiber proximal end extends a distance from the first transitory connector distal end.

15. The instrument of claim 14 further comprising:
a second transitory connector having a second transitory connector distal end and a second transitory connector proximal end;
a superior arm of the second transitory connector having a superior arm barb; and
an inferior arm of the second transitory connector having an inferior arm barb.

16. The instrument of claim 15 further comprising:
an illumination optic fiber having an illumination optic fiber distal end and an illumination optic fiber proximal end.

17. The instrument of claim 16 wherein the illumination optic fiber is fixed within the second transitory connector.

18. The instrument of claim 17 wherein the illumination optic fiber proximal end extends a distance from the second transitory connector distal end.

19. The instrument of claim 18 wherein the illumination optic fiber is fixed within the one-piece handle.

20. The instrument of claim 19 wherein the illumination optic fiber distal end is coplanar with the one-piece handle distal end.
